(19) United States
(12) Patent Application Publication
(10) Pub. No.: US 2006/0224149 A1
(43) Pub. Date: Oct. 5, 2006

(54) Apparatus and method positioning a therapeutic probe with respect to a therapeutic target

(75) Inventor: Ron Hillely, Zichron Yaakov (IL)

Correspondence Address: Martin Moynihan;Prtsi Inc, PO Box 16466, Arlington, VA, 22215, US

(73) Assignee: Galil Medical Ltd., Yokneam (IL)

(21) Appl. No.: 10/553,631

(22) PCT Filed: Apr. 21, 2004
(86) PCT No.: PCT/IL04/00340

§ 371 Date: Oct. 19, 2005

Related U.S. Application Data
(60) Provisional application No. 60/464,120, filed on Apr. 21, 2003.

(51) Int. Cl. A61B 18/02

(52) U.S. Cl. 606/21

(57) ABSTRACT

The present invention is of a device and method for positioning a plurality of therapeutic probes with respect to a treatment target within a patient. The method comprises the steps of a) utilizing standard imaging modalities to direct an orientation probe to the target, b) rigidly affixing to the inserted orientation probe a template comprising one or more probe guides, thereby orienting template and probe guides with respect to the target, then c) inserting one or more therapeutic probes through probe guides of the template and into the patient, thereby guiding the therapeutic probes to the treatment target.

### FIELD AND BACKGROUND OF THE INVENTION

The present invention relates to a device and method for positioning a therapeutic probe with respect to a treatment target within a patient. More particularly, the present invention is of an device and method for positioning one or more therapeutic probes, such as cryoprobes, with respect to a tumor, lesion, or other treatment target in a patient, by utilizing standard imaging modalities to direct an orientation probe to the target, then rigidly affixing to the orientation probe a template comprising one or more probe guides, thereby orienting template and probe guides with respect to the target, then inserting one or more therapeutic probes through probe guides of the template and into the patient, thereby guiding the therapeutic probes to the treatment target.

Therapeutic probes are used for tissue ablation in a variety of surgical contexts. Probes deliver RF energy, microwave energy, laser light, and other forms of energy designed to destroy tumors or other unwanted body tissues. Alternatively, therapeutic probes are used to destroy tissue by cryogenic cooling. The prior art embodiments presented in detail hereinbelow are primarily directed to the guidance of cryoprobes for cryoablation of tissues in a patient, yet it is noted that the invention is not limited to this exemplary embodiment. Indeed, the invention is relevant to guiding the placement of therapeutic probes of various sorts, including RF probes, laser probes, microwave probes, and any variety of therapeutic probes usable for percutaneous treatment of body tissues.

The need for accurately positioning cryoprobes with respect to a treatment target, and prior art methodologies for doing so, are similar to requirements and solutions for positioning probes delivering heat energy or electrical energy to a treatment site. Thus, the following discussion of prior art with respect to delivery of cryoprobes to a treatment site may be taken as representative of the problem of positioning of percutaneous probes in general.

Cryoablation of pathological tissues is an increasingly popular method of treatment for such conditions as cancers of prostate, liver, and kidney, and for treating benign prostate hyperplasia (“BPH”). Cryoablation of pathological tissues is typically accomplished by utilizing imaging modalities such as x-ray, ultrasound, CT, and MRI to identify a locus for ablative treatment, then inserting one or more cryoprobes into that selected treatment locus, and cooling the treatment heads of those cryoprobes sufficiently to cause the tissues surrounding the treatment heads to reach cryoablation temperatures, typically below about-40° C. The tissues thus cooled are thereby caused to loose their functional and structural integrity. Cancerous cells cease growing and multiplying, and cryoablated tumor tissue material, whether from malignant tumors or from benign growths, is subsequently absorbed by the body. Cryoablation may thus be used to treat malignant tumors of the prostate, the liver, the kidneys, and other organs, and to reduce prostate volume in cases of BPH.

The principle danger and disadvantage of cryosurgical ablative treatment of tissues is the danger of partially or completely destroying the functional and structural integrity of non-pathological tissues proximate to the treatment locus, thereby having a deleterious effect on the health and quality of life of the treated patient.

Various devices and methods have been proposed to enable cryoablation of pathological prostate tissue while limiting damage to non-pathological tissue. In particular, a variety of methods and devices for accurate placement of cryoprobes are used in cryoablation, so as to successfully concentrate the cooling effect of such cryoprobes at or near pathological tissue and minimize unwanted cooling of non-pathological tissue, are known in the art.

An example is provided by U.S. Pat. No. 6,142,991 to Schatzberger. Schatzberger describes a high resolution cryosurgical method and device for treating a patient's prostate, including the steps of (a) introducing a plurality of cryosurgical probes to the prostate, the probes having a substantially small diameter, the probes being distributed across the prostate, so as to form an outer arrangement of probes adjacent the periphery of the prostate and an inner arrangement of probes adjacent the prostatic urethra; (b) producing an ice-ball at the end of each of said cryosurgical probes, so as to locally freeze a tissue segment of the prostate. Schatzberger's apparatus includes (a) a plurality of cryosurgical probes of small diameter, the probes being for insertion into the patient's organ, the probes being for producing ice-balls for locally freezing selected portions of the organ; (b) a guiding element including a net of apertures for inserting the cryosurgical probes therethrough; and (c) an imaging device for providing a set of images, the images being for providing information on specific planes located at specific depths within the organ, each of said images including a net of marks being correlated to the net of apertures of the guiding element, wherein the marks represent the locations of ice-balls which may be formed by the cryosurgical probes when introduced through said apertures of the guiding element to said distinct depths within the organ.

Thus, Schatzberger's method and apparatus enable a surgeon to place a set of cryoablation probes within a prostate with relatively high accuracy, and to operate those probes to ablate selected tissues while avoiding, to a large extent, inadvertent and undesirable ablation of healthy tissues near the ablation site. In practice, Schatzberger's guiding element is typically used to introduce a plurality of straight cryoprobes, in parallel, into a cryoablation target area.

However, Schatzberger's device and technique present an important disadvantage. Schatzberger's guiding element, containing a plurality of apertures used to guide insertion of individual cryoprobes, is connected, in a predetermined positional relationship, to an ultrasound probe which provides images which in principle are useable for determining which apertures should be used to guide cryoprobes, and to what depth those cryoprobes should be inserted. In actual therapeutic practice, the ultrasound probe and the guiding element are rigidly connected to a stepper stabilizer device which is connected to the patient's bed. In this arrangement, the longitudinal axis of the ultrasound probe and the apertures of the guiding element are substantially parallel, and the device permits movement of its elements (the guiding element, the ultrasound probe, and the therapeutic probes) only forward and backward along this common axis. The active head of the ultrasound probe can also be twisted around this principal axis.

In use, Schatzberger's guiding element is thus typically fixed in place, connected to an ultrasound probe which in turn is inserted into the rectum of a patient, and the whole rigidly connected to a stepper stabilizing device, prior to actual insertion of any of the therapeutic cryoprobes. However, clinical experience has shown that the ultrasound images provided by the rectal ultrasound probe, while useful for determining appropriate depths for the inserted cryoprobes, are not ideally suited for determining in advance which aperture of Schatzberger's guiding element is lined up with (i.e., pointing towards) the center of the cryoablation target. Thus, proper placement of the initially inserted cryoprobes requires some guesswork. If it turns out (as seen by ultrasound observation) that the first inserted probe is not well directed towards the cryoablation target, the surgeon has no choice but to retract the probe and re-insert it through a different aperture, or else to re-adjust the entire stepper-stabilizer device orientation. The position and orientation of Schatzberger's guiding element is fixed, because the guiding element has as fixed positional relationship to an inserted rectal ultrasound probe and to the stepper stabilizer device. Therefore it is not possible to adjust the position of Schatzberger's guiding element based on the actual observed position of a first inserted cryoprobe, once that cryoprobe has been inserted.

Thus, there is a widely felt need for, and it would be highly advantageous to have, a device and method for guiding therapeutic probes, such as cryoprobes, towards a treatment target, wherein an initial orientation probe can be freely and conveniently inserted into a target while being observed under imaging modalities selected by a surgeon according to his convenience and according to the therapeutic requirements of the case, where the surgeon is free to observe the insertion of the orientation probe from a variety of angles and is further free to select the insertion angle of the orientation probe according to his convenience and according to the therapeutic requirements of the case, and wherein the surgeon's movement is unrestricted and his field of vision unobstructed during this initial insertion process, yet which device and method provide means for accurately guiding a plurality of therapeutic probes to selected positions with respect to the treatment target, once the initial orientation probe is correctly placed.

### SUMMARY OF THE INVENTION

According to one aspect of the present invention there is provided a method for guiding a therapeutic probe to a treatment target within the body of a patient, comprising (a) inserting an orientation probe into the body of a patient and positioning the orientation probe so that the orientation probe has a known spatial relationship to the treatment target; (b) rigidly affixing to the orientation probe a template which comprises at least one probe guide operable to guide movement of a therapeutic probe inserted therethrough in a controlled direction, the controlled direction being aligned with the treatment target when the template is rigidly affixed to the inserted orientation probe; and (c) inserting at least one therapeutic probe through the at least one probe guide into the body of a patient, thereby guiding the inserted therapeutic probe to the treatment target.

According to further features in preferred embodiments of the invention described below, the method further comprises operating the at least one therapeutic probe, when positioned at the treatment target, to ablate at least a portion of the treatment target.

According to further features in preferred embodiments of the invention described below, the method further comprises utilizing an imaging modality to position the orientation probe so that the orientation probe has a known spatial relationship to the treatment target. The utilized imaging modality may be selected from a group consisting of ultrasound imaging, CT scanning, X-ray imaging, fluoroscope imaging, and MRI.

The method further comprises positioning the orientation probe so that a distal portion of the orientation probe is positioned within the treatment target.

According to further features in preferred embodiments of the invention described below, the at least one therapeutic probe is a cryoprobe operable to cryoablate tissue at the treatment target. The cryoprobe may be operable to be cooled by Joule-Thomson cooling and heated by Joule-Thomson heating.

Preferably, the template comprises an elastic pressure clamp utilizable to rigidly affix the template to the orientation probe. The elastic pressure clamp may be operable to be released by pressure on a handle of the template.

Preferably the template comprises a plurality of probe guides and the method further comprises inserting a plurality of therapeutic probes into the body of a patient, each through one of the plurality of probe guides.

Preferably, the orientation probe comprises a set of marks useable to measure a distance of insertion of the orientation probe through the template, and the at least one therapeutic probe comprises a set of marks useable to measure a distance of insertion of the at least one therapeutic probe through the template, and the method further comprises inserting the at least one therapeutic probe to a distance having a selected relationship to a measured distance of insertion of the orientation probe through the template.

The at least one probe guide may be an aperture in the template, the aperture being designed and constructed to constrain a therapeutic probe inserted therethrough to movement along a predetermined axis.

Preferably, the template further comprises a plurality of the apertures.

More preferably, template comprises a plurality of mutually parallel apertures.

The axis of the aperture may be perpendicular to a surface of the template, or the template may comprise a plurality of apertures having axes oriented in a common direction. The common direction may be perpendicular to a surface of the template, and may be substantially parallel to a longitudinal axis of the orientation probe when the orientation probe is affixed to the template.

The orientation probe may be a therapeutic probe, which may be a cryoprobe.

According to further features in preferred embodiments of the invention described below, the at least one probe guide is of fixed orientation with respect to the template.

According to still further features in preferred embodiments of the invention described below, the at least one probe guide is of variable orientation with respect to the template.

The template may comprise a plurality of probe guides whose axes are oriented so as to concentrate distal portions of a plurality of probes inserted therethrough, or a plurality of probe guides whose axes are oriented so as to disperse distal portions of a plurality of probes inserted therethrough.

Preferably, the template is constructed of ertacetal resin, and may comprise circular markings indicating boundaries of tissue destruction expected when ablation probes are inserted through probe guides of the template into a body of a patient and the ablation probes are activated to ablate body tissues under standardized conditions.

The template may be rigidly affixed to the orientation probe by pressure clamping, which may be accomplished by additional steps of (d) squeezing a handle of the template to cause separation of two portions of the template; (e) positioning the separated portions of the template around the orientation probe, after the orienatation probe has been inserted according to the procedure of step (a) above; (f) releasing the handle of the template, thereby allowing the separated portions of the template to spring back towards each other, thereby seizing a portion of the orientation probe between the separated portions, thereby rigidly affixing the template to the orientation probe.

At least a portion of the treatment target may be within a prostate, or within a liver, or within a kidney.

According to another aspect of the present invention there is provided a device for guiding a therapeutic probe to a treatment target within the body of a patient, comprising a template operable to be rigidly affixed to an orientation probe inserted in the body of a patient, which template comprises at least one probe guide operable to constrain movement of a therapeutic probe inserted therethrough to movement in a controlled direction, such that if a straight orientation probe is inserted into the body of a patient in such manner that a distal portion of the orientation probe is positioned within the treatment target, and the template is rigidly affixed to the orientation probe, then a therapeutic probe being inserted into the body of a patient through the at least one probe guide will be constrained to move towards the treatment target.

The device may further comprise the orientation probe, which may be a therapeutic probe, which may be a cryoprobe.

The orientation probe may be a solid probe devoid of differentiated internal parts.

Preferably, the device further comprises at least one therapeutic probe operable to be inserted into the body of a patient through the at least one probe guide. The therapeutic probe may be an ablation probe operable to ablate tissue at the treatment site, such as a cryoprobe operable to cryoablate tissue at the treatment target. The cryoprobe may be operable to be cooled by Joule-Thomson cooling and to be heated by Joule-Thomson heating.

Preferably the template comprises an elastic pressure clamp utilizable to rigidly affix the template to the orientation probe.

The elastic pressure clamp may be operable to be released by pressure on a handle of the template.

Preferably, the template comprises a plurality of probe guides and a plurality of therapeutic probes, each operable to be inserted through one of the plurality of probe guides.

Preferably, the orientation probe comprises as set of marks useable to measure a distance of insertion of the orientation probe through the template and the at least one therapeutic probe comprises a set of marks useable to measure a distance of insertion of the at least one therapeutic probe through the template.

According to still further features in the described preferred embodiments, the at least one probe guide is an aperture in the template, the aperture is operable to constrain a therapeutic probe inserted therethrough to move only along a predetermined movement axis, the axis having a constant orientation with respect to the template.

Preferably, the template further comprises a plurality of the apertures, whose axes may be mutually parallel.

According to still further features in the described preferred embodiments, the predetermined axis is perpendicular to a face of the template.

Preferably, the template comprises a plurality of apertures whose axes are oriented in a common direction, which may be perpendicular to a surface of the template.

Still preferably, the common direction is substantially parallel to a direction at which the orientation probe extends from the template, when the orientation probe is affixed to the template, which direction is preferably perpendicular to a surface of the template.

Preferably, the orientation probe is a therapeutic probe such as a cryoprobe.

The at least one probe guide may be of fixed or of variable orientation with respect to the template.

Preferably the template is operable to be rigidly affixed to the orientation probe by pressure clamping, and further operable to grip the orientation probe between two separable parts of a gripping aperture, and further operable to release the orientation probe when a squeezing pressure is applied to a handle of the template.

The present invention successfully addresses the shortcomings of the presently known configurations by providing a device and method for guiding therapeutic probes, such as cryoprobes, towards a treatment target, wherein an orientation probe can be freely and conveniently inserted into a target while being observed under imaging modalities selected by a surgeon according to his convenience and according to the therapeutic requirements of the case, the surgeon being free to observe the insertion of the orientation probe from a variety of angles and to freely choose an insertion angle for the orientation probe, the surgeon's movement being unrestricted and his field of vision unobstructed during this initial insertion process, yet which device and method are operable to guide a plurality of additional probes to selected positions in or near the treatment target, once the initial orientation probe is correctly placed.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic of an exemplary cryoprobe, according to the methods of prior art;

FIG. 2 is a simplified schematic of a manifold structure connecting a plurality of cryosurgical probes to a common gas source, according to the methods of prior art;

FIG. 3 is a simplified schematic of an alternative configuration of a pre-cooling element, according to the methods of prior art;

FIG. 4 is a simplified schematic of an apparatus comprising an ultrasound probe and a guiding element for guiding insertion of a plurality of cryoprobes into a patient's body, according to the methods of prior art;

FIG. 5 is a simplified schematic showing a method of use of the apparatus presented in FIG. 4, according to the methods of prior art;

FIG. 6 is a simplified schematic showing a further step in the use of the apparatus presented in FIG. 4, according to the methods of prior art;

FIG. 7 is a schematic representation of a template for guiding therapeutic probes to a treatment target, according to an embodiment of the present invention;

FIG. 8 is a simplified flow chart of a procedure for positioning a plurality of therapeutic probes at a treatment target, utilizing the template presented in FIG. 7, according to an embodiment of the present invention;

FIG. 9 is an adaptation of a photographic image of a template showing details of the passage of a plurality of therapeutic probes through guiding elements of the template, according to an embodiment of the present invention;

FIG. 10 is an adaptation of a photographic image of a template and of a plurality of probes passing therethrough, according to an embodiment of the present invention; and

FIG. 11 is an adaptation of a photographic image of a template in use during an actual surgical procedure, according to an embodiment of the present invention.

### DESCRIPTION OF THE PREFERRED EMBODIMENTS

The present invention is of a device and method for positioning a plurality of therapeutic probes with respect to a treatment target within a patient. More particularly, the present invention is of an device and method for positioning one or more therapeutic probes, such as cryoprobes, with respect to a tumor, lesion, or other treatment target in a patient, by utilizing standard imaging modalities to direct an orientation probe to the target, then rigidly affixing to the orientation probe a template comprising one or more probe guides, thereby orienting template and probe guides with respect to the target, then inserting one or more therapeutic probes through probe guides of the template and into the patient, thereby guiding the therapeutic probes to the treatment target.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrase “heat-exchanging configuration” is used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when passed through a Joule-Thomson heat exchanger. As is well known in the art, when gasses such as argon, nitrogen, air, krypton, C0₂, CF₄, xenon, and N₂O, and various other gasses pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which have the property of becoming hotter when passed through a Joule-Thomson heat exchanger. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating.

In discussion of the various figures described hereinbelow, like numbers refer to like parts.

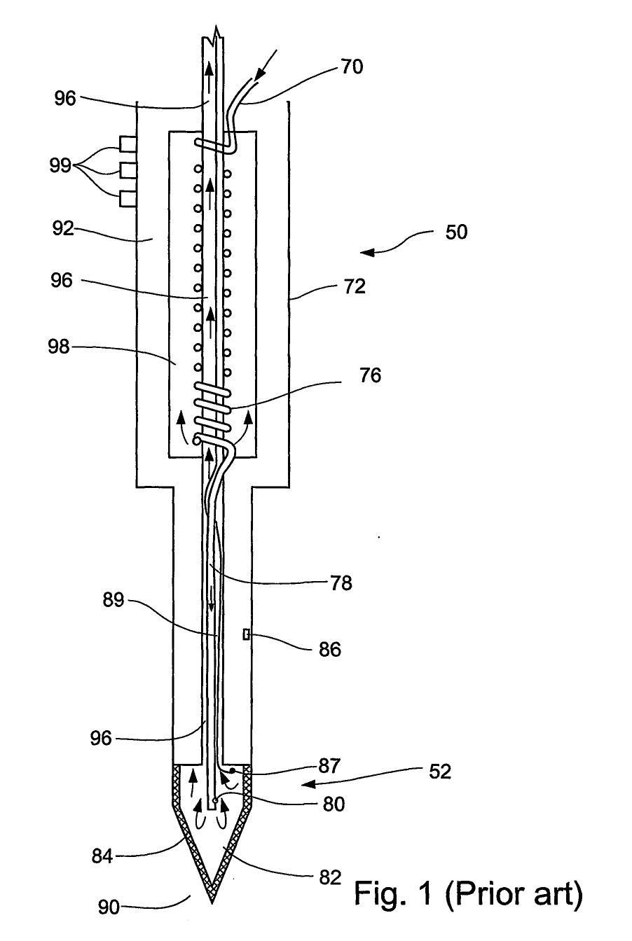

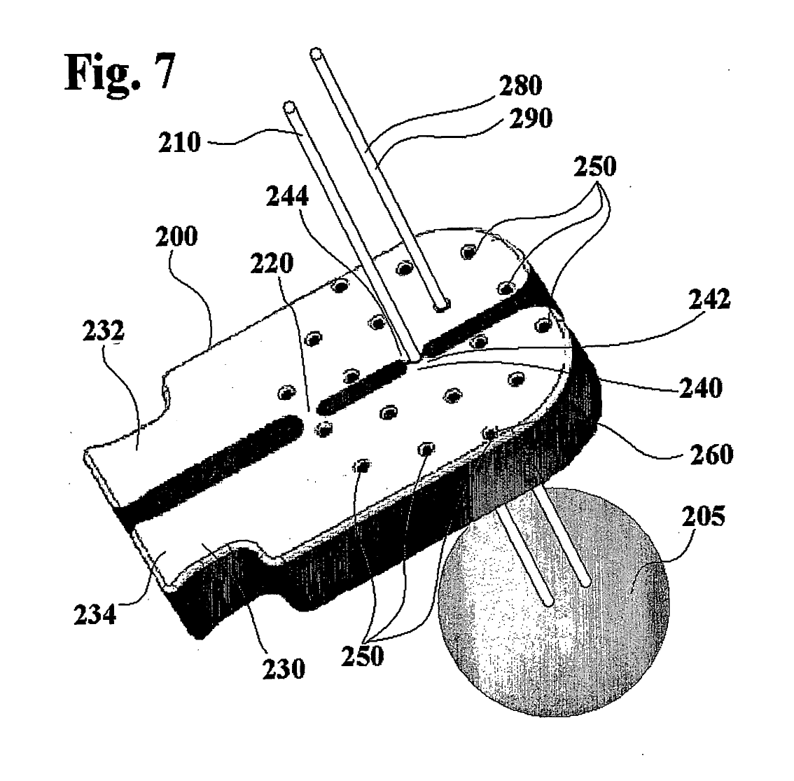

For purposes of better understanding the present invention, as illustrated in FIGS. 7-11 of the drawings, reference is first made to the construction and operation of conventional (i.e., prior art) cryosurgery apparatus and treatment method as illustrated in FIGS. 1-6.

Referring to FIGS. 1-3, a cryosurgical apparatus according to methods of prior art includes a plurality of cryosurgical probes.

FIG. 1 presents a simplified schematic of an exemplary cryoprobe, according to the methods of prior art.

FIG. 1 presents a cryoprobe 50 having an operating tip 52 including a Joule-Thomson cooler for freezing a patient's tissue and a holding member 72 for holding by a surgeon. As shown in FIG. 1, operating tip 52 includes at least one passageway 78 extending therethrough for providing gas of high pressure to orifice 80 located at the end of operating tip 52, orifice 80 being for passage of high pressure cooling gas therethrough, so as to cool operating tip 52 and produce an ice-ball at its end 90.

When a high pressure cooling gas such as argon expands through orifice 80 it may liquefy, so as to form a cryogenic pool within chamber 82 of operating tip 52, which cryogenic pool effectively cools surface 84 of operating tip 52. Surface 84 of operating tip 52 is preferably made of a heat conducting material such as metal so as to enable the formation of an ice-ball at end 90 thereof.

Alternatively, a high pressure heating gas such as helium may be used for heating operating tip 52 via a reverse Joule-Thomson process, so as to enable treatment by cycles of cooling-heating, and further for preventing sticking of the probe to the tissue when extracted from the patient's body, and to enable fast extraction when so desired.

When a high pressure heating gas such as helium expands through orifice 80 it heats chamber 82, thereby heating surface 84 of operating tip 52.

Operating tip 52 includes at least one evacuating passageway 96 extending therethrough for evacuating gas from operating tip 52 to the atmosphere.

As shown in FIG. 1, holding member 72 may include a heat exchanger for pre-cooling the gas flowing through passageway 78. Specifically, the upper portion of passageway 78 may be in the form of a spiral tube 76 wrapped around evacuating passageway 96, the spiral tube being accommodated within a chamber 98. Thus, gas evacuated through passageway 96 may pre-cool the incoming gas flowing through spiral tube 76.

As further shown in FIG. 1, holding member 72 may include an insulating body 92 for thermally insulating the heat exchanger from the external environment.

Furthermore, operating tip 52 may include at least one thermal sensor 87 for sensing the temperature within chamber 82, the wire 89 of which extending through evacuating passageway 96 or a dedicated passageway (not shown). Probe 50 may further comprise one or more external thermal sensors 86, preferably placed at some distance from operating tip 52, operable to report on temperatures induced in surrounding tissues by cooling of operating tip 52.

In addition, holding member 72 may include a plurality of switches 99 for manually controlling the operation of probe 50 by a surgeon. Such switches may provide functions such as on/off, heating, cooling, and predetermined cycles of heating and cooling by selectively and controllably communicating incoming passageway 70 with an appropriate external gas container including a cooling or a heating gas.

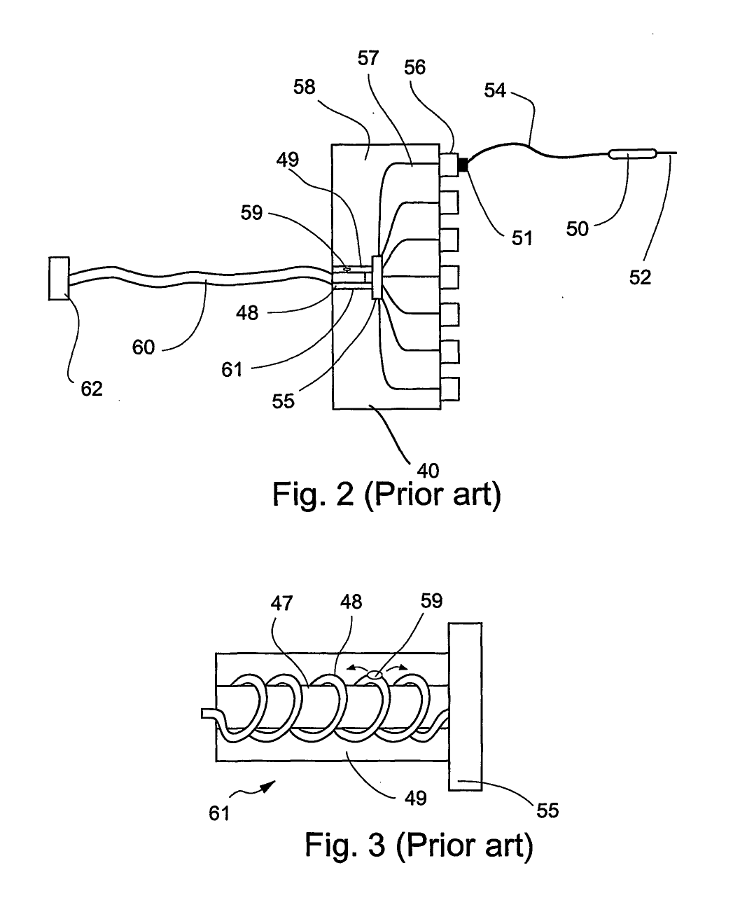

Attention is now drawn to FIG. 2, which presents a simplified schematic of a gas distribution module connecting a plurality of cryosurgical probes 50 to a common gas source, according to the methods of prior art.

FIG. 2 presents a gas distribution module 40, wherein each of cryosurgical probes 50 is connected via a flexible connecting line 54 to a connecting site 56 on a housing element 58, preferably by means of a linking element 51. Cryosurgical probes 50 may be detachably connected to connecting sites 56.

Preferably, evacuating passageway 96 extends through connecting line 54, such that the outgoing gas is evacuated through an opening located at linking element 51 or at any other suitable location, e.g., manifold 55, see below. Preferably, line 54 further includes electrical wires for providing electrical signals to the thermal sensor and switches (not shown).

Each of cryosurgical probes 50 is in fluid communication with a manifold 55 received within a housing 58, manifold 55 being for distributing the incoming high pressure gas via lines 57 to cryosurgical probes 50.

As shown, housing 58 is connected to a connector 62 via a flexible cable 60 including a gas tube (not shown), connector 62 being for connecting the apparatus to a high pressure gas source and an electrical source.

The apparatus further includes electrical wires (not shown) extending through cable 60 and housing 58 for providing electrical communication between the electrical source and cryosurgical probes 50.

Preferably, housing 58 includes a pre-cooling element, generally designated as 61, for pre-cooing the high pressure gas flowing to cryosurgical probes 50. Preferably, pre-cooling element 61 is a Joule-Thomson cooler, including a tubular member 48 received within a chamber 49, tubular member 48 including an orifice 59 for passage of high pressure gas therethrough, so as to cool chamber 49, thereby cooling the gas flowing through tubular member 48 into manifold 55.

Attention is now drawn to FIG. 3, which presents an alternative configuration of a pre-cooling element 61 according to the methods of prior art, wherein tubular member 48 is in the form of a spiral tube wrapped around a cylindrical element 47, so as to increase the area of contact between tubular member 48 and the cooling gas in chamber 49.

According to yet another configuration (not shown), housing 58 includes a first tubular member for supplying a first high pressure gas to manifold 55, and a second tubular member for supplying a second high pressure gas to pre-cooling element 61. Any combination of gases may be used for cooling and/or heating the gases flowing through such tubular members.

Alternatively, a cryogenic fluid such as liquid nitrogen may be used for pre-cooling the gas flowing through housing 58. Alternatively, an electrical pre-cooling element may used for pre-cooling the gas.

Preferably, thermal sensors (not shown) may be located within cable 60 and manifold 55 for measuring the temperature of gas flowing therethrough.

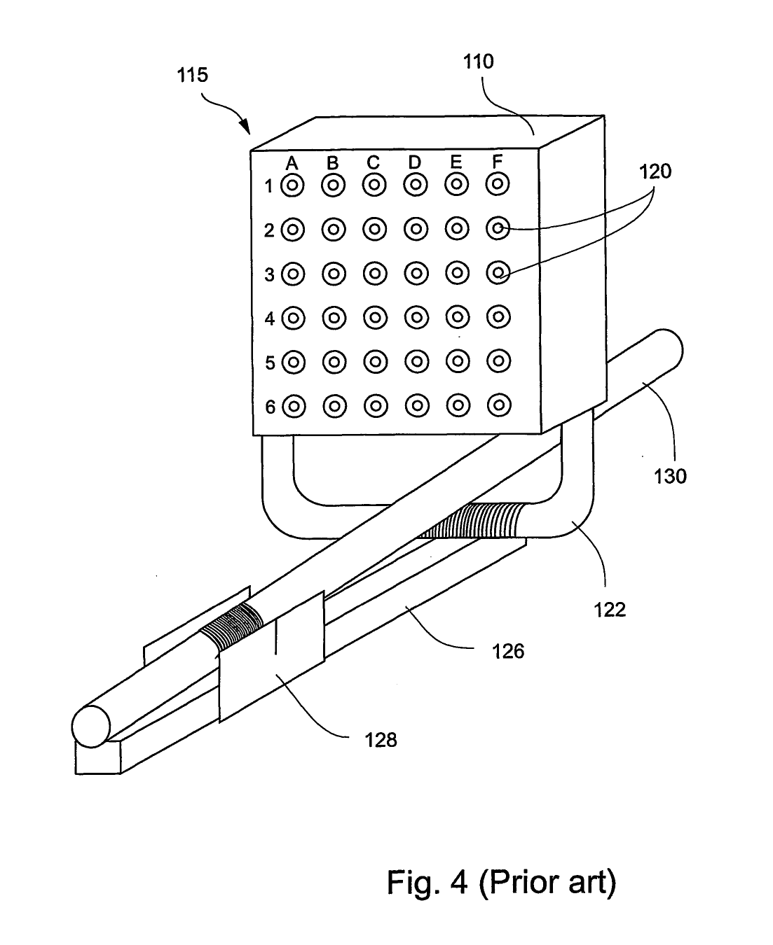

Attention is now drawn to FIGS. 4-6, which present a prior art method and apparatus utilizing an imaging device to form a three-dimensional grid of the patient's treated organ, e.g., prostate, the three dimensional grid serves for providing information on the three dimensional shape of the organ. Each of a set of cryosurgical probes is then inserted to a specific depth within the organ according to the information provided by the grid.

FIG. 4 is a simplified schematic of an apparatus comprising an ultrasound probe and a guiding element for guiding insertion of a plurality of cryoprobes into a patient's body, according to the methods of prior art.

As shown in FIG. 4, an ultrasound probe 130 is provided for insertion into the patient's rectum, ultrasound probe 130 being received within a housing element 128. A guiding element 115 is connected to housing element 128 by means of a connecting arm 126. As shown, guiding element 115 is in the form of a plate 110 having a net of apertures 120, each aperture serves for insertion of a cryosurgical probe therethrough. Preferably, the distance between each pair of adjacent apertures 120 is between about 2 millimeters and about 5 millimeters.

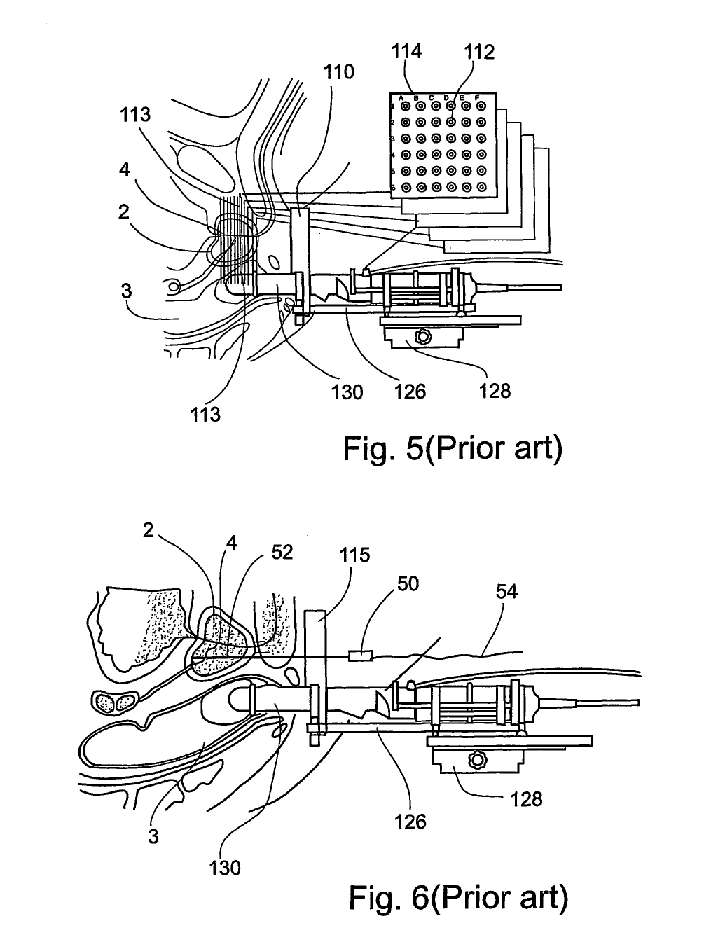

Attention is now drawn to FIG. 5, which is a simplified schematic showing a method of use of the apparatus presented in FIG. 4.

As shown in FIG. 5, ultrasound probe 130 is introduced to a specific depth 113 within the patient's rectum 3. A net of marks 112 is provided on the obtained ultrasound image 114, the net of marks 112 on image 114 being accurately correlated to the net of apertures 120 on guiding element 115.

Thus, marks 112 on image 114 sign the exact locations of the centers of ice-balls which may be formed at the end of the cryosurgical probes inserted through apertures 120 to the patient's prostate 2, wherein image 114 relates to a specific depth of penetration 113 of the cryosurgical probes into the prostate 2.

As shown in FIG. 5, ultrasound probe 130 is gradually introduced to various depths 113 of rectum 3, thereby producing a set of images 114, wherein each image relates to a respective depth of penetration into the prostate 2. Thus, each of images 114 relates to a specific plane perpendicular to the axis of penetration of the cryosurgical probes.

The set of images 114 provides a three dimensional grid of the prostate. Such three-dimensional grid is then used for planning the cryosurgical procedure.

For example, the introduction of a cryosurgical probe along a given axis of penetration to a first depth may effectively destroy a prostatic tissue segment, while introduction of the probe to a second depth may severely damage the prostatic urethra.

Since the ice-ball is locally formed at the end of the cryosurgical probe, each probe may be introduced to a specific depth so as to locally provide an effective treatment to a limited portion of the prostate while avoiding the damaging of non-prostatic or prostatic tissues located at other depths of penetration.

Attention is now drawn to FIG. 6, which is a simplified schematic presenting a further step in the use of the apparatus presented in FIG. 4, according to the methods of prior art.

FIG. 6 shows the insertion of an operating tip 52 of a cryosurgical probe 50 through an aperture of guiding element 115 into the prostate 2 of a patient.

In typical use, a plurality of cryosurgical probes are sequentially inserted through apertures 120 of guiding element 115 into the patient's prostate, wherein each probe is introduced to a specific depth, thereby providing substantially local effective treatment to distinct segments of the prostatic tissue while avoiding the damaging of other prostatic or non-prostatic tissue segments.

Preferably, each of the cryosurgical probes includes a scale for indicating the depth of penetration into the prostate.

Thus, it may be seen that the prior art apparatus and methods presented by FIGS. 1-6 enable diagnostic mapping of areas to be treated within a prostate, and further enable guiding a plurality of cryogenic probes into a prostate in such a manner that the cryogenic probes are placed according to the planned treatment areas so mapped.

As may be seen from FIG. 6, all cryoprobes used must be introduced into the body of the patient through apertures 120 of guiding element 115. Guiding element 115 must thus be in place before insertion of cryoprobes begins.

Preferred embodiments of the present invention may now be described. It is noted, however, that the aforementioned prior art context is here described for exemplary purposes only. The invention disclosed herein is not limited to the exemplary context. Embodiments of the present invention may be used for cryoablation of organs other than the prostate. Cryoprobes dissimilar to cryoprobe 50 presented in FIG. 1 may be utilized in embodiments of the present invention, on condition that they are capable of cooling tissues to cryoablation temperatures. Therapeutic ablation probes other than cryoprobes, such as probes delivering RF energy, electrical resistance heating energy, microwave energy, laser light energy, or other forms of probes may be used in alternative embodiments of the present invention. Therapeutic probes other than ablation probes, such as probes for measuring temperatures or otherwise ascertaining local conditions within a patient's tissues, or probes providing therapeutic imaging modalities, may be guided to a treatment target utilizing embodiments of the present invention.

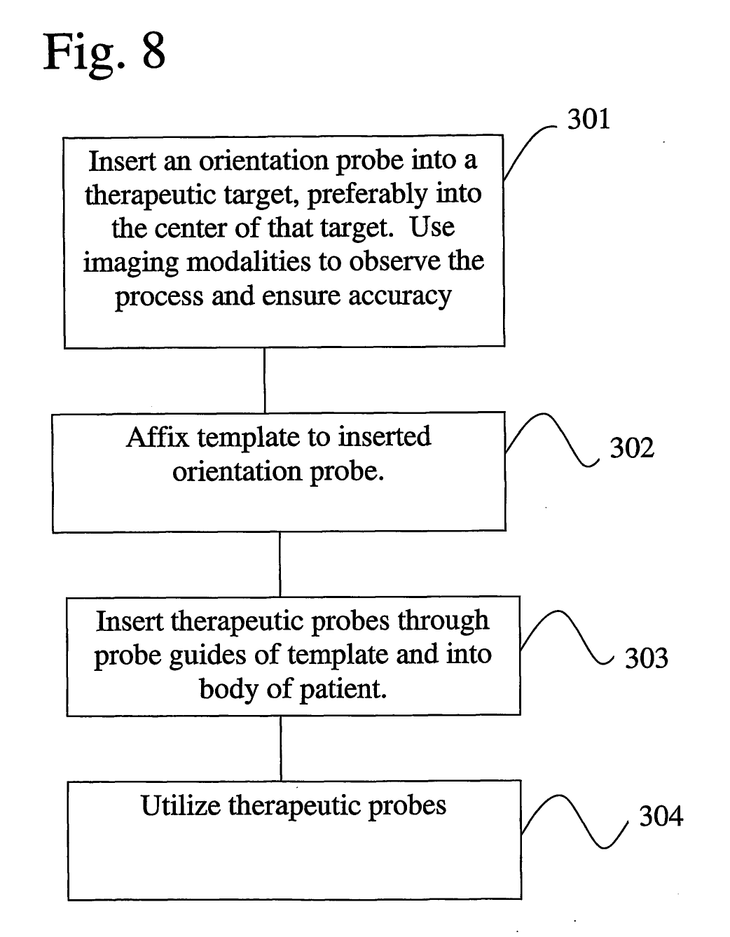

Attention is now drawn to FIG. 7, which is a schematic representation of a template useable for guiding therapeutic probes to a treatment target, according to a preferred embodiment of the present invention. Discussion of FIG. 7 refers also to FIG. 8, which is a simplified flow chart of a therapeutic procedure utilizing the device presented by FIG. 7, according to a preferred embodiment of the present invention.

FIG. 7 presents a template 200 useable to accurately position a plurality of therapeutic probes with respect to a selected surgical target. In this sense template 200 is similar to prior art guiding element 115 presented by FIGS. 4-6. In contrast, however, to the guiding element 115, template 200 of the present invention is designed and constructed to enable a surgeon to insert a first probe (referred to herein as an “orientation probe”) into a surgical target prior to placement of template 200 in the surgical area.

Clinical experience has shown that once a template such as guiding element 115 is properly aimed at a target, prior art guiding element 115 is useful to enable placement of a plurality of probes in a selected spatial relationship one to another, so as to achieve a desired therapeutic effect, e.g., a common cryogenic ice ball of selected size and shape. However, experience has also shown that with prior art guiding element 115 fixed in position (that is, once ultrasound probe 130 is inserted in the rectum of a patient), a surgeon may experience difficulty in determining which (if any) aperture 120 of guiding element 115 is accurately aimed at the center of the treatment target. Information provided by the ultrasound images produced by probe 130 may be insufficient to permit this determination, and selection of an aperture 120 for initial insertion of therapeutic probes may be more a process of successive approximation than of accurate predetermination.

In contrast, template 200 of FIG. 7 is designed to allow initial placement of a first probe, orientation probe 210, before template 200 is placed near the patient. Thus, in a first step of a recommended procedure, (step 301 of FIG. 8), a surgeon inserts orientation probe 210 into a selected position with respect to a treatment target 205. Typically, he inserts the orientation probe to the center of an ablation target such as a tumor.) Template 200 is unconnected to positioning probe 210 during step 301, consequently the surgeon is enabled to insert probe 210 to a desired position with respect to a treatment target, without restriction by or interference from template 200. At this stage of the procedure the surgeon has an unrestricted view of his patient and the operating area, is free to use any convenient combination of imaging modalities to guide his insertion of positioning probe 210, and is free to insert positioning probe 210 at whatever angle seems to him most likely to place the tip of probe 210 at a desired portion (typically, the center) of target 205. Preferably, the surgeon will utilize medical imaging modalities to verify accurate positioning of probe 210 with respect to treatment target 205. Imaging modalities utilized by a surgeon for this purpose may include ultrasound imaging, CT scans, X-ray and fluoroscope imaging, MRI, and other imaging modalities.

Orientation probe 210 may be a therapeutic probe such as a cryoprobe, or, alternatively, may be a solid probe devoid of internal functioning parts, whose sole function is that of orienting template 200 with respect to a treatment target, as explained below.

After orientation probe 210 is positioned as desired with respect to a treatment target, template 200 is then rigidly attached to probe 210 at a fixed orientation with respect to probe 210. This is step 302 of FIG. 8. Template 200 remains external to the patient. In a presently preferred embodiment shown in FIG. 7, template 200 attaches to probe 210 in a manner which guarantees that template 200 will be perpendicular to probe 210. In alternative embodiments, template 200 attaches to probe 210 at a selected fixed angle other than 90° . It is recommended that the surgeon attach template 200 to probe 210 as close as possible to the point of entry of probe 210 into the body of the patient.

In an exemplary embodiment shown in FIG. 7, template 200 attaches to probe 210 by pressure clamping. Template 200 comprises a flexible region 220 having elastic physical characteristics. Flexible region 220 is preferably constructed of an elastic material. Alternatively, flexible region 220 may be constructed of a moveable joint and an external or internal spring, such as a plastic or metallic spring, to enhance the clamping effect.

In the embodiment of the present invention shown in FIG. 7, sides 232 and 234 of handle 230 may be squeezed together, bending flexible region 220, and causing separation of sides 242 and 244 of gripping aperture 240. With sides 242 and 244 thus separated, template 200 may be placed around inserted orientation probe 210 in such a way that orientation probe 210 passes between the separated halves 242 and 244 of gripping aperture 240. Once template 200 is so positioned, sides 232 and 234 of handle 230 may be released.

When sides 232 and 234 of handle 230 are released, elasticity of flexible region 220 causes sides 242 and 244 of gripping area 240 to grip and hold positioning probe 210, which passes between them. Thus, with handle 230 released, gripping area 240 grips probe 210, effectively clamping template 200 to orientation probe 210, thereby fixing the position and orientation of template 200 with respect to probe 210, and consequently also with respect to treatment target 205. In a recommended embodiment of the present invention, probe 210 is a straight probe whose distal portion is positioned at the center of treatment target 205 during step 301 of the procedure, and gripping area 240 of probe 200 is designed and constructed to grip and hold template 200 at right angles to probe 210. Consequently, template 200, once clamped to probe 210, is perpendicular to straight orientation probe 210, which points directly towards treatment target 205.

In a third step of a recommended procedure, step 303 of FIG. 8, probe guides 250 of template 200 are used to guide insertion of at least one therapeutic probe 280 into target 205. In a recommended embodiment, a plurality of probe guides 250 are provided in template 200, and are used at step 303 to introduce a plurality of therapeutic probes 280 into the therapeutic target.

Probe guides 250 may be any configuration operable to guide insertion of therapeutic probes 280 into the body of a patient. Thus, probe guides 250 may be individually orientable to selected orientations. Yet, in a presently preferred embodiment shown in Figure. 7, probe guides 250 are simply apertures 260, perpendicular to the surface of template 200 and parallel to each other, similar to apertures 120 of FIG. 4. Alternatively, apertures 120 may traverse template 200 at a selected angle other than perpendicular. Apertures 260 are sized to conform to the external diameter of therapeutic probes 280 to be inserted therethrough, and thus can serve to guide and direct the movement of therapeutic probes 280 as therapeutic probes 280 are inserted into the body of a patient. The angle at which apertures 260 traverse template 200, and consequently the angle to which movement of a therapeutic probe 280 through an aperture 260 is constrained, is referred to in the following as the “axis” or “axis of movement” of that aperture 260. Preferably, insertion distance markings are provided on orientation probe 210 and on therapeutic probes 280 and may be used by a surgeon to control depth of penetration of probes 280 in comparison to the depth of penetration of probe 210, whose depth of penetration into the therapeutic target area is known and was observed, using imaging modalities, during step 301 of the procedure presented in FIG. 8.

At optional step 304 of FIG. 8, inserted therapeutic probes 280, and optionally probe 210 if probe 210 is also a therapeutic probe 280, are used to treat (e.g., to ablate) tissues in the target area. In a preferred embodiment, probes 280 are cryoprobes 290 similar to probe 50 presented in FIG. 1, and at step 304 cooling gas is supplied to Joule-Thomson orifices within probes 290, thereby cooling probes 290 to cryoablation temperatures and cryoablating tissues at the therapeutic target site.

In this manner, template 200 may be used to guide ablation of tumors of the prostate, of the kidney, of the liver, and of various other organs susceptible to percutaneous laparoscopic ablation.

Currently recommended dimensions for template 200 designed for guidance of 2 mm probes are length 72 mm, height 44 mm and thickness 10 mm. Currently recommended dimensions for template 200 designed for guidance of 3 mm probes are length 76 mm, height 52 mm, and thickness 10 mm. Template 200 must be thick enough for probe guides 250 to provide accurate control of the direction of therapeutic probes 280 passing therethrough, yet thin enough to enable adequate penetration of therapeutic probes 280 into a body of a patient. Template 200 is preferably constructed of Delrin (ertacetal resin), or similar plastic materials, which may be sterilized using ethylene oxide sterilization, or of Teflon, or of metals of various sorts.

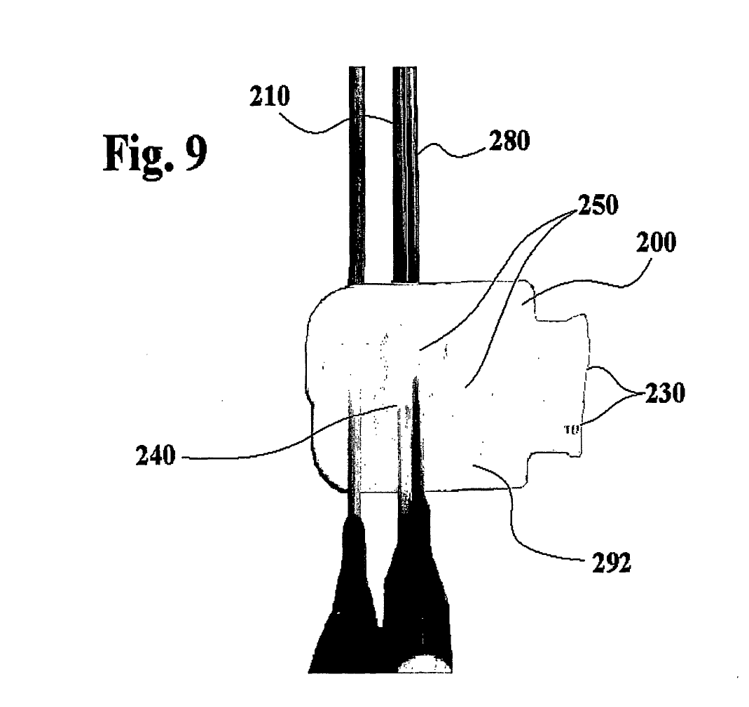

Attention is now drawn to FIGS. 9-11, each of which presents a template 200 gripping an orientation probe 210. In the embodiment shown in these figures, orientation probe 210 is also a therapeutic probe 280, namely a cryoprobe 290. FIGS. 9-11 also show a plurality of additional therapeutic probes 280, which in this embodiment are cryoprobes 290, passing through probe guides 250 of template 200.

FIG. 9 is an adaptation of a photographic image of a template 200, slowing details of the passage of a plurality of probes 280 through probe guides 250 of template 200, according to an embodiment of the present invention. Marked circles 292 around each probe guide 250 are provided to indicate an estimated effective ablation area around a tip of each inserted probe, which markings may be useful in helping the user to design accurately a desirable distribution of probes for use for a particular target shape.

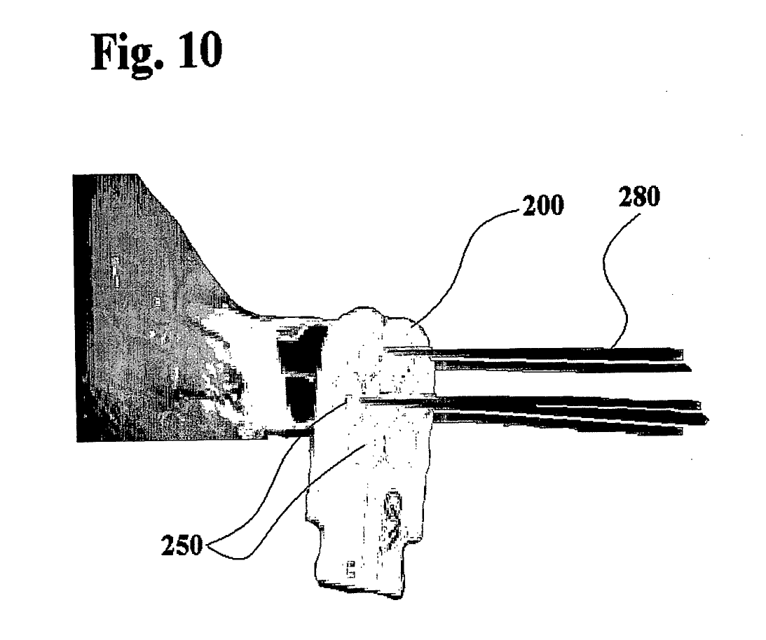

FIG. 10 is also an adaptation of a photograph of a template 200 and a plurality of probes passing therethrough, according to an embodiment of the present invention. FIG. 10 demonstrates how probe guides 250 of template 200 constrain the position and orientation of a plurality of therapeutic probes 280 passing through template 200, so that the distal operating tips of probes 280, at some distance from template 200, are grouped and positioned in a desired configuration. The embodiment presented by FIG. 10 is a presently preferred configuration, in which probe guides 250 are embodied as apertures 260 in template 200, which apertures are perpendicular to template 200 and parallel to each other. Alternative configurations include parallel apertures 260 which are not perpendicular to the surface of template 200, non-parallel apertures 260 oriented so as to further concentrate the distal operating portions of inserted therapeutic probes in the target area, and non-parallel apertures designed to disperse the distal operating portions of inserted therapeutic probes 280 in the target area to a selected degree.

In a recommended embodiment, template 200 is a disposable template designed for one-time use. A plurality of disposable templates 200 may be made available to a surgeon, in a variety of configurations, thereby providing to the surgeon a choice of the number of available probe guides 250, of their proximity, of their diameter, of the degree to which they concentrate or disperse the operating tips of probes inserted therethrough, of the types of therapeutic probe for which they are appropriate, of the type of methodology used to fix template 200 to probe 210, and various other selectable characteristics.

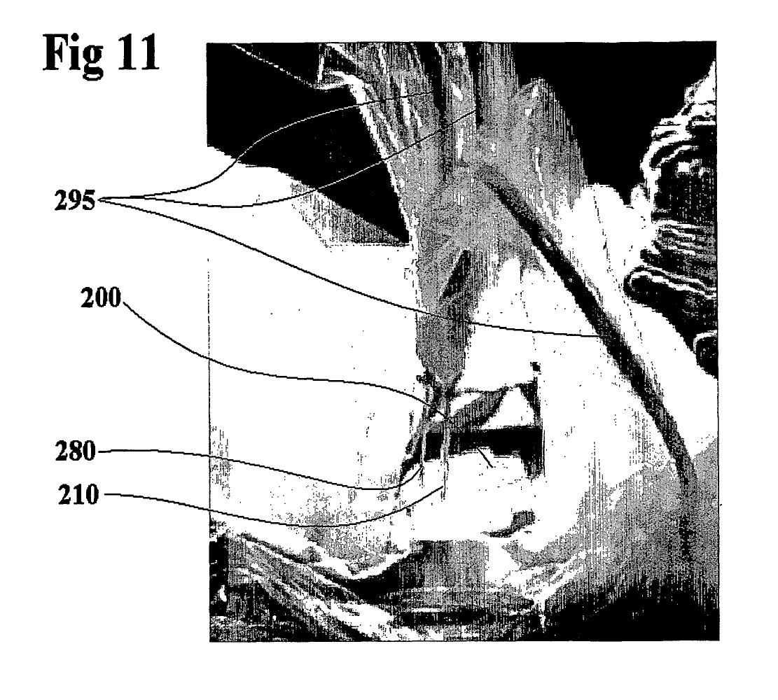

Attention is now drawn to FIG. 11, which presents an adaptation of a photograph image of a template 200 in use during an actual surgical procedure. Three cryoprobes 290 may be seen in. FIG. 11, each connected to a gas supply line 295 operable to supply high pressure cooling gas to a Joule-Thomson orifice within each cryoprobe 290. Preferably, gas lines 295 are also operable to supply compressed heating gas to probes 290, thereby providing for heating of probes 290 to facilitate disengagement of probes 290 at the conclusion of the cooling phase of a cryoablation procedure.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

In the claims:

1. A method for guiding a therapeutic probe to a treatment target within the body of a patient, comprising:
(a) inserting an orientation probe into the body of a patient and positioning said orientation probe so that said orientation probe has a known spatial relationship to said treatment target;
(b) rigidly affixing to said orientation probe a template which comprises at least one probe guide operable to constrain movement of a therapeutic probe inserted therethrough in a controlled direction, said controlled direction being aligned with said treatment target when said template is rigidly affixed to said inserted orientation probe;
(c) inserting at least one therapeutic probe through said at least one probe guide into the body of a patient,
thereby guiding said inserted therapeutic probe to said treatment target.

2. The method of claim 1, further comprising operating said at least one therapeutic probe, when positioned at said treatment target, to ablate at least a portion of said treatment target.

3. The method of claim 1, further comprising utilizing an imaging modality to position said orientation probe so that said orientation probe has a known spatial relationship to said treatment target.

4. The method of claim 3, wherein said utilized imaging modality is selected from a group consisting of ultrasound imaging, CT scanning, X-ray imaging, fluoroscope imaging, and MRI.

5. The method of claim 1, further comprising positioning said orientation probe so that a distal portion of said orientation probe is positioned within said treatment target.

6. The method of claim 1, wherein said at least one therapeutic probe is a cryoprobe operable to cryoablate tissue at said treatment target.

7. The method of claim 6, wherein said cryoprobe is operable to be cooled by Joule-Thomson cooling.

8. The method of claim 7, wherein said cryoprobe is further operable to be heating by Joule-Thomson heating.

9. The method of claim 1, wherein said template comprises an elastic pressure clamp utilizable to rigidly affix said template to said orientation probe.

10. The method of claim 9, wherein said elastic pressure clamp is operable to be released by pressure on a handle of said template.

11. The method of claim 1, wherein said template comprises a plurality of probe guides.

12. The method of claim 11, further comprising inserting a plurality of therapeutic probes into the body of a patient, each through one of said plurality of probe guides.

13. The method of claim 1, wherein said orientation probe comprises a set of marks useable to measure a distance of insertion of said orientation probe through said template.

14. The method of claim 13, wherein said at least one therapeutic probe comprises a set of marks useable to measure a distance of insertion of said at least one therapeutic probe through said template.

15. The method of claim 14, further comprising inserting said at least one therapeutic probe to a distance having a selected relationship to a measured distance of insertion of said orientation probe through said template.

16. The method of claim 1, wherein said at least one probe guide is an aperture in said template, said aperture being designed and constructed to constrain a therapeutic probe inserted therethrough to movement along a predetermined axis.

17. The method of claim 16, wherein said template further comprises a plurality of said apertures.

18. The method of claim 17, wherein said template comprises a plurality of mutually parallel apertures.

19. The method of claim 16, wherein said axis of said aperture is perpendicular to a surface of said template.

20. The method of claim 17, wherein said template comprises a plurality of apertures having axes oriented in a common direction.

21. The method of claim 20, wherein said common direction is perpendicular to a surface of said template.

22. The method of claim 20, wherein said common direction is substantially parallel to a longitudinal axis of said orientation probe when said orientation probe is affixed to said template.

23. The method of claim 22, wherein said common direction is perpendicular to a surface of said template.

24. The method of claim 1, wherein said orientation probe is a therapeutic probe.

25. The method of claim 1, wherein said orientation probe is a cryoprobe.

26. The method of claim 1, wherein said at least one probe guide is of fixed orientation with respect to said template.

27. The method of claim 1, wherein said at least one probe guide is of variable orientation with respect to said template.

28. The method of claim 11, wherein said template comprises a plurality of probe guides whose axes are oriented so as to concentrate distal portions of a plurality of probes inserted therethrough.

29. The method of claim 11, wherein said template comprises a plurality of probe guides whose axes are oriented so as to disperse distal portions of a plurality of probes inserted therethrough.

30. The method of claim 1, wherein said template is constructed of ertacetal resin.

31. The method of claim 1 wherein said template further comprises circular markings indicating boundaries of tissue destruction expected when ablation probes are inserted through probe guides of said template into a body of a patient and said ablation probes are activated to ablate body tissues under standardized conditions.

32. The method of claim 1, wherein said template is rigidly affixed to said orientation probe by pressure clamping.

33. The method of claim 32, wherein said pressure clamping is accomplished by additional steps of:
(d) squeezing a handle of said template to cause separation of two portions of said template;
(e) positioning said separated portions of said template around said orientation probe, after said orienatation probe has been inserted according to the procedure of step (a);
(f) releasing said handle of said template, thereby allowing said separated portions of said template to spring back towards each other, thereby seizing a portion of said orientation probe between said separated portions; thereby rigidly affixing said template to said orientation probe.

34. The method of claim 1, wherein at least a portion of said treatment target is within a prostate.

35. The method of claim 1, wherein at least a portion of said treatment target is within a liver.

36. The method of claim 1, wherein at least a portion of said treatment target is a within a kidney.

37. A device for guiding a therapeutic probe to a treatment target within the body of a patient, comprising a template operable to be rigidly affixed to an orientation probe inserted in the body of a patient, which template comprises at least one probe guide operable to constrain movement of a therapeutic probe inserted therethrough to movement in a controlled direction, such that if an orientation probe is inserted into the body of a patient in such manner that a distal portion of said orientation probe is positioned within said treatment target, and said template is rigidly affixed to said orientation probe, then a therapeutic probe being inserted into the body of a patient through said at least one probe guide will be constrained to move towards said treatment target.

38. The device of claim 37, further comprising said orientation probe.

39. The device of claim 38, wherein said orientation probe is a therapeutic probe.

40. The device of claim 39, wherein said therapeutic probe is a cryoprobe.

41. The device of claim 38, wherein said orientation probe is a solid probe devoid of differentiated internal parts.

42. The device of claim 37, further comprising at least one therapeutic probe operable to be inserted into the body of a patient through said at least one probe guide.

43. The device of claim 42, wherein said therapeutic probe is an ablation probe operable to ablate tissue at said treatment site.

44. The device of claim 42, wherein said therapeutic probe is a cryoprobe operable to cryoablate tissue at said treatment target.

45. The device of claim 42, wherein said cryoprobe is operable to be cooled by Joule-Thomson cooling.

46. The device of claim 45, wherein said cryoprobe is further operable to be heating by Joule-Thomson heating.

47. The device of claim 37, wherein said template comprises an elastic pressure clamp utilizable to rigidly affix said template to said orientation probe.

48. The device of claim 47, wherein said elastic pressure clamp is operable to be released by pressure on a handle of said template.

49. The device of claim 37, wherein said template comprises a plurality of probe guides.

50. The device of claim 49, further comprising a plurality of therapeutic probes, each operable to be inserted through one of said plurality of probe guides.

51. The device of claim 37, wherein said orientation probe comprises as set of marks useable to measure a distance of insertion of said orientation probe through said template.

52. The device of claim 51, wherein said at least one therapeutic probe comprises a set of marks useable to measure a distance of insertion of said at least one therapeutic probe through said template.

53. The device of claim 37, wherein said at least one probe guide is an aperture in said template, said aperture is operable to constrain a therapeutic probe inserted therethrough to move only along a predetermined movement axis, said axis having a constant orientation with respect to said template.

54. The device of claim 53, wherein said template further comprises a plurality of said apertures.

55. The device of claim 54, wherein said template comprises a plurality of apertures whose axes are mutually parallel.

56. The device of claim 53, wherein said predetermined axis is perpendicular to a face of said template.

57. The device of claim 54, wherein said template comprises a plurality of apertures whose axes are oriented in a common direction.

58. The device of claim 57, wherein said common direction is perpendicular to a surface of said template.

59. The device of claim 57, wherein said common direction is substantially parallel to a direction at which said orientation probe extends from said template, when said orientation probe is affixed to said template.

60. The device of claim 59, wherein said common direction is perpendicular to a surface of said template.

61. The device of claim 37, wherein said orientation probe is a therapeutic probe.

62. The device of claim 37, wherein said orientation probe is a cryoprobe.

63. The device of claim 37, wherein said at least one probe guide is of fixed orientation with respect to said template.

64. The device of claim 37, wherein said at least one probe guide is of variable orientation with respect to said template.

65. The device of claim 49, wherein said template comprises a plurality of probe guides whose axes are oriented so as to concentrate distal portions of a plurality of probes inserted therethrough.

66. The device of claim 49, wherein said template comprises a plurality of probe guides whose axes are oriented so as to disperse distal portions of a plurality of probes inserted therethrough.

67. The device of claim 37, wherein said template is constructed of ertacetal resin.

68. The device of claim 37 wherein said template further comprises circular markings indicating boundaries of expected tissue destruction when ablation probes are inserted through probe guides of said template into a body of a patient and said probes are activated to ablate body tissues under standardized conditions.

69. The device of claim 37, wherein said template is operable to be rigidly affixed to said orientation probe by pressure clamping.

70. The device of claim 69, operable to grip said orientation probe between two separable parts of a gripping aperture, and further operable to release said orientation probe when a squeezing pressure is applied to a handle of said template.
